SEQUENCE LISTING (6 sequences)

SEQ ID NO: 1 - DNA, 21 bp, artificial sequence
  synthetic oligonucleotide
caaatccttacgtgccgcgaa

SEQ ID NO: 2 - DNA, 24 bp, artificial sequence
  synthetic oligonucleotide
ccattaagctcccaacagtaacct

SEQ ID NO: 3 - DNA, 993 bp, artificial sequence
  synthetid oligonucleotide
aagactcaaacttagaaacttgaattagatgtggtattcaaatccttagctgccgcgaagacacagacagcccccgtaagaacccacgaagcaggcgaagttcattgttctcaacattctagctgctcttgctgcatttgctctggaattcttgtagagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataaactctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactgtggaaactctttatgttagatatatttctctaggttactgttgggagcttaatggtagaaacttccttggtttcatgattaaactcttttttttcctg

SEQ ID NO: 4 - DNA, 701 bp, artificial sequence
  synthetic oligonucleotide
cccccatggctaggcagtgctccnnnnatgaatattntgacagtttgttgcatgncntgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataagctctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaannnnngnaaactctttatgttagatatatt

SEQ ID NO: 5 - DNA, 639 bp, artificial sequence
  synthetic oligonucleotide
caaatccttacgtgccgcgaagacacagacagcccccgtgtgaccaattcagtgaaaggaacgaatgcgattctctggacctgtttgggactgagcttaataatttctttggcagttttcgtgctaatgtttttgctaaggaagataagctctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactgtggaaactctttatgttagatatatttctctaggttactgttgggagcttaatgg

SEQ ID NO: 6 - DNA, 668 bp, artificial sequence
  synthetic oligonucleotide
gttctcaacattctagctgctcttgctgcatttgctctggaattcttgtagagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatactcctcctctaacatgtcagcgttattgtaatgcaagatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactgtggaaactctttatgttagatatatt

(19) United States
(12) Patent Application Publication
(10) Pub. No.: US 2012/0213768 A1
(43) Pub. Date: Aug. 23, 2012

(54) Diagnostic and Therapeutic Uses for B Cell Maturation Antigen

(75) Inventors: SangKon Oh, Baltimore, MD (US); Maria Virginia Pascual, Dallas, TX (US); Gerard Zurawski, Midlothian, TX (US); Jacques F. Banchereau, Montclair, NJ (US)

(73) Assignee: BAYLOR RESEARCH INSTITUTE, Dallas, TX (US)

(21) Appl. No.: 13/397,932

(22) Filed: Feb. 16, 2012

Related U.S. Application Data
(60) Provisional application No. 61/444,732, filed on Feb. 19, 2011.

(51) Int. Cl. A61K 39/395; G01N 33/566; G01N 21/25; G01N 21/64; G01N 27/72; A61K 38/02; A61P 29/00; A61P 17/00; A61P 37/08; A61P 11/06; A61P 3/10; A61P 1/16; A61P 9/00; A61P 1/00; A61P 37/04; A61K 31/713; C07H 21/04; C07K 14/00; A61P 19/02; C12Q 1/68

(52) U.S. Cl. 424/130.1; 435/6.11; 435/7.24; 514/1.1; 514/18.7; 514/1.7; 424/184.1; 514/44 A; 536/23.1; 530/350; 514/16.7; 514/16.6

(57) ABSTRACT

Biomarkers and therapies against autoimmune diseases, including systemic lupus erythematosus (SLE) are described herein. The present invention is based on the discovery of B cell maturation antigen (BCMA) and BCMA variant expression on SLE monocytes that can be directly associated with disease activity. The findings of the present invention enable the design of monoclonal antibodies or recombinant proteins that can block BCMA and BCMA variants as well as BCMA-bound APRIL.

### CROSS-REFERENCE TO RELATED APPLICATIONS

This application is a non-provisional application of U.S. provisional patent application No. 61/444,732 filed on Feb. 19, 2011 and entitled “Diagnostic and Therapeutic Uses for B Cell Maturation Antigen” the entire contents of which is incorporated herein by reference.

### STATEMENT OF FEDERALLY FUNDED RESEARCH

This invention was made with U.S. Government support under Contract No. P50 AR055503 awarded by the National Institutes of Health (NIH). The government has certain rights in this invention.

### TECHNICAL FIELD OF THE INVENTION

The present invention relates in general diagnosis and therapy of systemic lupus erythematosus (SLE), and more particularly, to B cell maturation antigen (BCMA) and BCMA variants expressed on SLE patient monocytes that could be used as biomarkers of SLE as well as targets for the design of new therapeutics for autoimmune diseases, including SLE.

### REFERENCE TO A SEQUENCE LISTING

A Sequence Listing is attached and incorporated herein. The Sequence Listing reflects the sequences set forth as originally filed with no new matter incorporated into the Specification.

### BACKGROUND OF THE INVENTION

Without limiting the scope of the invention, its background is described in connection with the discovery of new biomarkers and therapies against autoimmune disorders, including systemic lupus erythematosus (SLE).

U.S. Patent Application No. 20090325196 (Dillon et al. 2009) provides a method of measuring the levels of BCMA in a biological sample, specifically upon the B cell surface. The diagnostic assays disclosed in the Dillon invention are useful in predicting an individual's likelihood of developing or currently suffering from an autoimmune disease, such as SLE, and for methods for treating an individual clinically diagnosed with an autoimmune disease. This diagnostic test serves to predict a patient's likelihood to respond to a specific drug treatment, in particular treatment with BLyS antagonists, either singly or in combination with other immune suppressive drugs.

U.S. Patent Application No. 20070249530 (Kelley and Patel, 2007) relates to polypeptides that inhibit APRIL and/or BAFF binding to BCMA, nucleic acid molecules encoding the polypeptides, and compositions comprising the polypeptides. The present invention also relates to methods for treating an immune-related disease or cancer using the polypeptides and compositions of the invention. The present invention also relates to methods for identifying inhibitors of APRIL/BAFF binding to BCMA and APRIL/BAFF signaling.

U.S. Pat. No. 6,774,106 issued to Theill and Yu (2004) describes interactions among APRIL/G70, AGP-3/BLYS, BCMA, and TACI and related methods of use and compositions of matter. The Theill patent provides a strategy for development of therapeutics for treatment of autoimmune diseases, and cancer, for prevention of transplant rejection. Disease states and disease parameters associated with APRIL and AGP-3 may be affected by modulation of BCMA or TACI; disease states and parameters associated with TACI can be affected by modulation of APRIL; disease states and parameters can be affected by modulation of any of TACI, BCMA, APRIL and AGP-3 by a single therapeutic agent or two or more therapeutic agents together.

### SUMMARY OF THE INVENTION

The present invention describes discovery of biomarkers and design of novel therapies against autoimmune diseases, including systemic lupus erythematosus (SLE). The present invention is based on the discovery of the B cell maturation antigen (BCMA) and BCMA variant expression on SLE monocytes is associated with disease activity and is therefore a valuable biomarker. The invention further describes the development of novel therapies comprising of monoclonal antibodies or recombinant proteins that can block BCMA and BCMA variants as well as BCMA-bound APRIL.

In one embodiment the instant invention discloses a method for diagnosing or detecting an autoimmune condition or disorder in a human subject comprising the steps of: identifying the human subject suspected of having the autoimmune condition or disorder; obtaining a biological sample from the subject, wherein the biological sample comprises monocytes; detecting the presence of a B cell maturation antigen (BCMA), BCMA variant, ligand or receptor bound BCMA, B-cell activating factor (BAFF), APRIL, TACI, BCMA-APRIL complex in or on the monocyte; and diagnosing or detecting the autoimmune condition or disorder based on the presence of the B cell maturation antigen (BCMA), BCMA variant, ligand or receptor bound BCMA, B-cell activating factor (BAFF), APRIL, TACI, BCMA-APRIL complex.

The autoimmune condition or disorder as disclosed hereinabove is selected from the group consisting of auto-inflammatory diseases of the skin, allergy, sclerosis, arteriosclerosis, multiple sclerosis, asthma, psoriasis, lupus, systemic lupus erythematosis, diabetes mellitus, myasthenia gravis, chronic fatigue syndrome, fibromyalgia, Crohn's disease, Hashimoto's thyroiditis, Grave's disease, Addison's disease, Guillian Barre syndrome, scleroderma, Sjogren's syndrome, multiple sclerosis, myasthenia gravis, Reiter's syndrome, Grave's disease, alopecia areata, anklosing spondylitis, antiphospholipid syndrome, auto-immune hemolytic anemia, auto-immune hepatitis, auto-immune lymphoproliferative syndrome (ALPS), auto-immune thrombocytopenic purpura (ATP), Behcet's disease, bullous pemphigoid, cardiomyopathy, celiac sprue-type dermatitis, chronic fatigue syndrome immune deficiency syndrome (CFIDS), chronic inflammatory demyelinating polyneuropathy, cicatricial pemphigold, cold agglutinin disease, limited sclerodema (CREST syndrome), Crohn's disease, Dego's disease, dermatomyositis, discoid lupus, essential mixed cryoglobulinemia, fibromyalgia-fibromyositis, Guillain-Barre syndrome, idiopathic pulmonary fibrosis, idiopathic thrombocytopenia purpura (ITP), IgA nephropathy, juvenile rheumatoid arthritis, Meniere's disease, mixed connective tissue disease, pemphigus vulgaris, polyarteritis nodosa, polychondritis, polyglancular syndromes, polymyalgia rheumatica, polymyositis, primary agammaglobulinemia, primary biliary cirrhosis, psoriasis, Raynaud's phenomenon, rheumatic fever, sarcoidosis, scleroderma, stiff-man syndrome, Takayasu arteritis, temporal arthritis/giant cell arthritis, ulcerative colitis, uveitis, vasculitis, vitiligo, and Wegener's granulomatosis. In a specific aspect the autoimmune condition or disorder is systemic lupus erythematosis (SLE). In one aspect the BCMA, BCMA variants or both are selected from the group consisting of SEQ ID NO: 3, SEQ ID NO: 4, SEQ ID NO: 5, SEQ ID NO: 6, and any combinations thereof. In another aspect the normal subject is a subject not suffering from any autoimmune condition or disorder. In yet another aspect the autoimmune disease is suspected to be systemic lupus erythematosis (SLE) and monocytes obtained from the human subject, but not normal human monocytes, express one or more BCMA isoforms selected from SEQ ID NO: 3, SEQ ID NO: 4, SEQ ID NO: 5, or SEQ ID NO: 6. In one aspect the step of detecting is selected from detecting the presence of nucleic acids or amino acids.

In a specific embodiment the present invention provides a method for diagnosing or detecting systemic lupus erythematosis (SLE) in a human subject comprising the steps of: obtaining monocytes from a subject suspected of having SLE; detecting a presence of a B cell maturation antigen (BCMA), BCMA variant, ligand or receptor bound BCMA, B-cell activating factor (BAFF), APRIL, TACI, BCMA-APRIL complex and modifications and combinations thereof in or on the monocytes; and diagnosing or detecting the SLE based on the presence of the BCMA, BAFF, APRIL, TACI, BCMA-APRIL complex in or on the monocytes of the subject suspected of having the SLE. In one aspect the normal subject is a subject not suffering from SLE or any other autoimmune condition or disorder. In another aspect the BCMA, BCMA variants or both are selected from the group consisting of SEQ ID NO: 3, SEQ ID NO: 4, SEQ ID NO: 5, SEQ ID NO: 6, and any combinations thereof. In yet another aspect the monocytes obtained from the human subject, but not normal human monocytes, express one or more BCMA isoforms selected from SEQ ID NO: 3, SEQ ID NO: 4, SEQ ID NO: 5, or SEQ ID NO: 6. In one aspect the step of detecting is selected from detecting the presence of nucleic acids or amino acids.

Another embodiment of the present invention relates to pharmaceutical composition comprising: a therapeutically effective amount of an agent comprising a polypeptide, a protein, a peptide, an antibody or variants and modifications thereof, wherein the agent is capable of binding or interacting and thereby blocking or inhibiting one or more actions of a B cell maturation antigen (BCMA), a BCMA variant, ligand or receptor bound BCMA, a B-cell activating factor (BAFF), an APRIL, a TACI or a BCMA-APRIL complex and an optional pharmaceutically acceptable carrier.

In one aspect the composition is adapted for use in a prophylaxis, treatment or both of one or more autoimmune disorders or conditions selected from the group consisting of auto-inflammatory diseases of the skin, allergy, sclerosis, arteriosclerosis, multiple sclerosis, asthma, psoriasis, lupus, systemic lupus erythematosis, diabetes mellitus, myasthenia gravis, chronic fatigue syndrome, fibromyalgia, Crohn's disease, Hashimoto's thyroiditis, Grave's disease, Addison's disease, Guillian Barre syndrome, scleroderma, Sjogren's syndrome, multiple sclerosis, myasthenia gravis, Reiter's syndrome, Grave's disease, alopecia areata, anklosing spondylitis, antiphospholipid syndrome, auto-immune hemolytic anemia, auto-immune hepatitis, auto-immune lymphoproliferative syndrome (ALPS), auto-immune thrombocytopenic purpura (ATP), Behcet's disease, bullous pemphigoid, cardiomyopathy, celiac sprue-type dermatitis, chronic fatigue syndrome immune deficiency syndrome (CFIDS), chronic inflammatory demyelinating polyneuropathy, cicatricial pemphigold, cold agglutinin disease, limited sclerodema (CREST syndrome), Crohn's disease, Dego's disease, dermatomyositis, discoid lupus, essential mixed cryoglobulinemia, fibromyalgia-fibromyositis, Guillain-Barre syndrome, idiopathic pulmonary fibrosis, idiopathic thrombocytopenia purpura (ITP), IgA nephropathy, juvenile rheumatoid arthritis, Meniere's disease, mixed connective tissue disease, pemphigus vulgaris, polyarteritis nodosa, polychondritis, polyglancular syndromes, polymyalgia rheumatica, polymyositis, primary agammaglobulinemia, primary biliary cirrhosis, psoriasis, Raynaud's phenomenon, rheumatic fever, sarcoidosis, scleroderma, stiff-man syndrome, Takayasu arteritis, temporal arthritis/giant cell arthritis, ulcerative colitis, uveitis, vasculitis, vitiligo, and Wegener's granulomatosis. In a specific aspect the autoimmune disorder or condition is systemic lupus erythematosis (SLE).

In another aspect the composition is administered by an oral route, a nasal route, topically or as an injection. In yet another aspect the injection is selected from the group consisting of subcutaneous, intravenous, intraperitoneal, intramuscular, and intravenous. In another aspect the composition may be administered as a combination therapy with a second therapeutic agent for treating an immune-related disease, a chemotherapeutic agent, a cytotoxic agent or any combinations thereof. In another aspect the composition is an antagonist, blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof. In another aspect the composition is an siRNA that is an antagonist, blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof. In one aspect the composition comprises a BCMA or BCMA variant antagonist.

In yet another embodiment the present invention describes an immunosuppressive composition for suppressing an immune response, for prophylaxis, for therapy or any combination thereof against systemic lupus erythematosis (SLE) in a human subject comprising: a) an agent comprising an antibody, a protein, a peptide, an antagonist or any combinations or modifications thereof, wherein the agent binds, blocks or inhibits a biological action of B cell maturation antigen (BCMA), a BCMA variant, ligand or receptor bound BCMA, a B-cell activating factor (BAFF), an APRIL, a TACI or a BCMA-APRIL complex and b) a pharmaceutically acceptable carrier, wherein the agent is comprised in an amount effective to suppress the immune response against SLE. In one aspect the composition is administered subcutaneously, intravenously, intraperitoneally, intramuscularly, and intravenously. In another aspect the composition blocks binding or inhibits the biological action of BCMA, BCMA variants or both are selected from the group consisting of SEQ ID NO: 3, SEQ ID NO: 4, SEQ ID NO: 5, SEQ ID NO: 6, and any combinations thereof. In yet another aspect the composition is an siRNA that is an antagonist, blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof. In another aspect the composition exerts its immunosuppressive action by blocking or inhibiting BCMA mediated activation signals to monocytes thereby inhibiting or modulating B cell responses. In another aspect the composition exerts its immunosuppressive action by blocking or inhibiting BCMA mediated trans-presentation of APRIL to B cells.

In one embodiment the present invention provides a vaccine composition comprising: a) an antibody, a protein, a peptide, an antagonist or any combinations or modifications thereof capable of binding, blocking or inhibiting a biological action of B cell maturation antigen (BCMA), a BCMA variant, ligand or receptor bound BCMA, a B-cell activating factor (BAFF), an APRIL, a TACI or a BCMA-APRIL complex and b) an optional pharmaceutically acceptable carrier or an adjuvant. In one aspect the composition suppresses an immune response, for prophylaxis, for therapy or any combination thereof against systemic lupus erythematosis (SLE) in a human subject. In another aspect the composition is administered by an oral route, a nasal route, topically or as an injection. In yet another aspect the injection is selected from the group consisting of subcutaneous, intravenous, intraperitoneal, intramuscular, and intravenous. In another aspect the composition may be administered as a combination therapy with a second therapeutic agent for treating an immune-related disease, a chemotherapeutic agent, a cytotoxic agent or any combinations thereof. In yet another aspect the composition is an antagonist, blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof. In one aspect the composition blocks or inhibits BCMA mediated activation signals to monocytes thereby inhibiting or modulating B cell responses. In another aspect the composition blocks or inhibits BCMA mediated trans-presentation of APRIL to B cells.

In another embodiment the present invention provides a method for treatment, prophylaxis or both against systemic lupus erythematosis (SLE) in a human subject comprising the steps of: identifying the human subject in need of the treatment, the prophylaxis or both against SLE and administering a therapeutically effective amount of an immunosuppressive composition or a vaccine comprising: a) an antibody, a protein, a peptide, an antagonist or any combinations or modifications thereof capable of binding, blocking or inhibiting a biological action of B cell maturation antigen (BCMA), a BCMA variant, ligand or receptor bound BCMA, a B-cell activating factor (BAFF), an APRIL, a TACI or a BCMA-APRIL complex and b) an optional pharmaceutically acceptable carrier or an adjuvant. In one aspect the composition or vaccine is administered by an oral route, a nasal route, topically or as an injection. In another aspect the injection is selected from the group consisting of subcutaneous, intravenous, intraperitoneal, intramuscular, and intravenous. In yet another aspect the composition or vaccine may be administered as a combination therapy with a second therapeutic agent for treating an immune-related disease, a chemotherapeutic agent, a cytotoxic agent or any combinations thereof. In one aspect the composition comprises a BCMA or BCMA variant antagonist.

In another aspect the composition or vaccine blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof. In a related aspect the composition or vaccine may be used in a prophylaxis, a therapy or both of one or more autoimmune disorders or conditions selected from the group consisting of auto-inflammatory diseases of the skin, allergy, sclerosis, arteriosclerosis, multiple sclerosis, asthma, psoriasis, lupus, systemic lupus erythematosis, diabetes mellitus, myasthenia gravis, chronic fatigue syndrome, fibromyalgia, Crohn's disease, Hashimoto's thyroiditis, Grave's disease, Addison's disease, Guillian Barre syndrome, scleroderma, Sjogren's syndrome, multiple sclerosis, myasthenia gravis, Reiter's syndrome, Grave's disease, alopecia areata, anklosing spondylitis, antiphospholipid syndrome, auto-immune hemolytic anemia, auto-immune hepatitis, auto-immune lymphoproliferative syndrome (ALPS), auto-immune thrombocytopenic purpura (ATP), Behcet's disease, bullous pemphigoid, cardiomyopathy, celiac sprue-type dermatitis, chronic fatigue syndrome immune deficiency syndrome (CFIDS), chronic inflammatory demyelinating polyneuropathy, cicatricial pemphigold, cold agglutinin disease, limited sclerodema (CREST syndrome), Crohn's disease, Dego's disease, dermatomyositis, discoid lupus, essential mixed cryoglobulinemia, fibromyalgia-fibromyositis, Guillain-Barre syndrome, idiopathic pulmonary fibrosis, idiopathic thrombocytopenia purpura (ITP), IgA nephropathy, juvenile rheumatoid arthritis, Meniere's disease, mixed connective tissue disease, pemphigus vulgaris, polyarteritis nodosa, polychondritis, polyglanular syndromes, polymyalgia rheumatica, polymyositis, primary agammaglobulinemia, primary biliary cirrhosis, psoriasis, Raynaud's phenomenon, rheumatic fever, sarcoidosis, scleroderma, stiff-man syndrome, Takayasu arteritis, temporal arthritis/giant cell arthritis, ulcerative colitis, uveitis, vasculitis, vitiligo, and Wegener's granulomatosis. In another aspect the composition is an antagonist, blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof. In yet another aspect the composition is an siRNA that is an antagonist, blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof. In another aspect the composition blocks or inhibits BCMA mediated activation signals to monocytes thereby inhibiting or modulating B cell responses. In another aspect the composition blocks or inhibits BCMA mediated trans-presentation of APRIL to B cells. In one aspect the composition comprises a BCMA or BCMA variant antagonist.

In another embodiment, the present invention includes a kit for detecting the presence of BCMA or a BCMA variant comprising one or more vials comprising a BCMA or BCMA variant detection agent adapted for detecting the presence of the BCMA or BCMA variant in or on a monocyte. In one aspect the BCMA or BCMA variant detection agent comprises a BCMA or BCMA binding agent. In another aspect the BCMA or BCMA variant detection agent comprises a BCMA or BCMA binding nucleic acid. In another aspect the BCMA or BCMA variant detection agent comprises a BCMA or BCMA binding antibody. In yet another aspect the BCMA or BCMA variant detection agent comprises a BCMA or BCMA cognate binding agent. In one aspect the BCMA or BCMA variant detection agent is defined as further comprising a chromophore, fluorophore, fluorescence resonance energy transfer (FRET) molecule, enzyme, metal particle, magnetic particle, a radiodense particle, beads, RFID, or a radioactive agent. The kit may further comprise instruction for obtaining monocytes and use of the agents for the detection of BMCA or BCMA variants in or on monocytes.

### BRIEF DESCRIPTION OF THE DRAWINGS

For a more complete understanding of the features and advantages of the present invention, reference is now made to the detailed description of the invention along with the accompanying figures and in which:

FIG. 1 is a plot showing the serum APRIL levels measured by ELISA (12 donors for each group);

FIG. 2 shows the monocytes from SLE patients expressing APRIL on their surface. Correlation between the % of APRIL+monocytes and SLEDAI is presented in the right panel;

FIGS. 3A and 3B show the SLE monocytes are more efficient than healthy monocytes for inducing naïve B cells to secrete Igs: FIG. 3A on day 12 of the co-culture of monocyte and B cells, total IgM, IgG, and IgA were measured by ELISA. Monocytes from 3 patients and 3 healthy donors were tested, FIG. 3B B cells were co-cultured with SLE monocytes in the presence of ctrl Ig, anti-BAFF, or TACI-Fc. % of CD38+CD20+ in CD19+ cells are presented;

FIG. 4 shows monocytes from SLE patients expressing full-length of BCMA and splicing variant 2, 3, and 4. PCR products were analyzed via TBE-PAGE;

FIGS. 5A-5C show that full-length BCMA present APRIL to B cells: FIG. 5A Surface BCMA expressions on full-length BCMA- and mock-transfectants, FIG. 5B BCMA presents APRIL on the cell surface, FIG. 5C Membrane-bound APRIL enhances B cell differentiation into PBs. Different numbers of transfectants, as indicated, were co-cultured with 4×10⁴ CD27-IgD+ B cells for 6 days. % of CD38⁺CD20⁻ cells in CD19⁺ cells; and

FIGS. 6A and 6B show that APRIL activates SLE monocytes. 1×10⁵ purified monocytes from PBMCs of healthy or patients were cultured overnight in the presence or absence of 4 μg/mL soluble APRIL. IL-6 (FIG. 6A) and IL-10 (FIG. 6B) by ELISA.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “Antigen Presenting Cells” (APC) refers to cells that are capable of activating T cells, and include, but are not limited to, certain macrophages, B cells and dendritic cells. “Dendritic cells” (DCs) refers to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells). These cells can be isolated from a number of tissue sources, and conveniently, from peripheral blood, as described herein. Dendritic cell binding proteins refers to any protein for which receptors are expressed on a dendritic cell. Examples include GM-CSF, IL-1, TNF, IL-4, CD40L, CTLA4, CD28, and FLT-3 ligand.

For the purpose of the present invention, the term “vaccine composition” is intended to mean a composition which can be administered to humans or to animals in order to induce an immune system response; this immune system response can result in a production of antibodies or simply in the activation of certain cells, in particular antigen-presenting cells, T lymphocytes and B lymphocytes. The vaccine composition can be a composition for prophylactic purposes or for therapeutic purposes, or both. As used herein, the term “antigen” refers to any antigen, which can be used in a vaccine, whether it involves a whole microorganism or a subunit, and whatever its nature: peptide, protein, glycoprotein, polysaccharide, glycolipid, lipopeptide, etc. They may be viral antigens, bacterial antigens, or the like; the term “antigen” also comprises the polynucleotides, the sequences of which are chosen so as to encode the antigens whose expression by the individuals to which the polynucleotides are administered is desired, in the case of the immunization technique referred to as DNA immunization. They may also be a set of antigens, in particular in the case of a multivalent vaccine composition which comprises antigens capable of protecting against several diseases, and which is then generally referred to as a vaccine combination, or in the case of a composition which comprises several different antigens in order to protect against a single disease, as is the case for certain vaccines against whooping cough or the flu, for example. The term “antibodies” refers to immunoglobulins, whether natural or partially or wholly produced artificially, e.g. recombinant. An antibody may be monoclonal or polyclonal. The antibody may, in some cases, be a member of one, or a combination immunoglobulin classes, including: IgG, IgM, IgA, IgD, and IgE.

As used herein, the term “autoimmune disorder” refers to a disease state in which a patient's immune system recognizes a self-antigen or auto antigen in that patient's organs or tissues as foreign and becomes activated. The activated immune cells that are directed against self or auto antigens can cause damage to the target organ or tissue or can damage other organs or tissues. The dysregulated immune cells secrete inflammatory cytokines that cause systemic inflammation or they can recognize self-antigens as foreign, thereby accelerating the immune response against self-antigens. Autoimmune disorders are often considered to be caused, at least in part, by a hypersensitivity reaction as found with allergies, because in both cases the immune system reacts to a substance that it normally would ignore.

Non-limiting examples of allergens or antigens that cause asthma include pollens (grass, tree and weeds), pet or insect dander, perfumes or scents, food (corn, wheat, eggs, milk, seafood, legumes, soy, tree nuts), fungi, seeds, nuts, alcohol, plant secretions, drugs (e.g., penicillin or other antibiotics, salicylates), insect bites (bees, wasps, spiders, flies, dust mites), natural and synthetic compounds (e.g., latex), animal products (fur, dander, wool), mold spores, and metal. Specific examples of compounds, peptides, carbohydrates, proteins, lipids and combinations thereof that may be attached to the antibodies and binding fragments of the present invention may be found in, e.g., the allergome database, e.g., www.allergome.org, relevant portions incorporated herein by reference.

Non-limiting examples of autoimmune diseases include: rheumatoid arthritis, auto-immune or auto-inflammatory diseases of the skin, allergy, sclerosis, arteriosclerosis, multiple sclerosis, asthma, psoriasis, lupus, systemic lupus erythematosis, diabetes mellitus, myasthenia gravis, chronic fatigue syndrome, fibromyalgia, Crohn's disease, Hashimoto's thyroiditis, Grave's disease, Addison's disease, Guillian Bane syndrome and scleroderma. “Auto-immune” refers to an adaptive immune response directed at self-antigens. “Auto-immune disease” refers to a condition wherein the immune system reacts to a “self” antigen that it would normally ignore, leading to destruction of normal body tissues. Auto-immune disorders include, e.g., Hashimoto's thyroiditis, pernicious anemia, Addison's disease, type 1 (insulin dependent) diabetes, rheumatoid arthritis, systemic lupus erythematosus, Sjogren's syndrome, multiple sclerosis, myasthenia gravis, Reiter's syndrome, and Grave's disease, alopecia areata, anklosing spondylitis, antiphospholipid syndrome, auto-immune hemolytic anemia, auto-immune hepatitis, auto-immune lymphoproliferative syndrome (ALPS), auto-immune thrombocytopenic purpura (ATP), Behcet's disease, bullous pemphigoid, cardiomyopathy, celiac sprue-type dermatitis, chronic fatigue syndrome immune deficiency syndrome (CFIDS), chronic inflammatory demyelinating polyneuropathy, cicatricial pemphigold, cold agglutinin disease, limited sclerodema (CREST syndrome), Crohn's disease, Dego's disease, dermatomyositis, discoid lupus, essential mixed cryoglobulinemia, fibromyalgia-fibromyositis, Guillain-Barre syndrome, idiopathic pulmonary fibrosis, idiopathic thrombocytopenia purpura (ITP), IgA nephropathy, juvenile rheumatoid arthritis, Meniere's disease, mixed connective tissue disease, pemphigus vulgaris, polyarteritis nodosa, polychondritis, polyglancular syndromes, polymyalgia rheumatica, polymyositis, primary agammaglobulinemia, primary biliary cirrhosis, psoriasis, Raynaud's phenomenon, rheumatic fever, sarcoidosis, scleroderma, stiff-man syndrome, Takayasu arteritis, temporal arthritis/giant cell arthritis, ulcerative colitis, uveitis, vasculitis, vitiligo, and Wegener's granulomatosis.

The term “adjuvant” refers to a substance that enhances, augments or potentiates the host's immune response to a vaccine antigen.

The term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated

As used herein, the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

As used in this application, the term “amino acid” means one of the naturally occurring amino carboxylic acids of which proteins are comprised. The term “polypeptide” as described herein refers to a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, the term “in vivo” refers to being inside the body. The term “in vitro” used as used in the present application is to be understood as indicating an operation carried out in a non-living system.

As used herein, the term “treatment ” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

As used herein, an “isolated nucleic acid molecule” refers to a nucleic acid molecule that is not integrated in the genomic DNA of an organism. For example, a DNA molecule that encodes a growth factor that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

As used herein, a “sample” or “specimen” is any mixture of macromolecules obtained from a person. This includes, but is not limited to, blood, plasma, urine, semen, saliva, lymph fluid, meningeal fluid, amniotic fluid, glandular fluid, and cerebrospinal fluid. This also includes experimentally separated fractions of all of the preceding. “Sample” also includes solutions or mixtures containing homogenized solid material, such as feces, cells, tissues, and biopsy samples. Samples herein include one or more that are obtained at any point in time, including diagnosis, prognosis, and periodic monitoring.

As used herein, the term “homology” refers to the extent to which two nucleic acids are complementary. There may be partial or complete homology. A partially complementary sequence is one that at least partially inhibits a completely complementary sequence from hybridizing to a target nucleic acid and is referred to using the functional term “substantially homologous.” The degree or extent of hybridization may be examined using a hybridization or other assay (such as a competitive PCR assay) and is meant, as will be known to those of skill in the art, to include specific interaction even at low stringency.

As used herein, the terms “protein”, “polypeptide” or “peptide” refer to compounds comprising amino acids joined via peptide bonds and are used interchangeably. An amino acid or “polypeptide” is a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, the term “hybridize” refers to any process by which a strand of nucleic acid binds with a complementary strand through base pairing. Hybridization and the strength of hybridization (i.e., the strength of the association between the nucleic acid strands) is impacted by such factors as the degree of complementary between the nucleic acids, stringency of the conditions involved, the melting temperature of the formed hybrid, and the G:C (or U:C for RNA) ratio within the nucleic acids.

The term “labeled” as used herein indicates that there is some method to visualize or detect the bound probe, whether or not the probe directly carries some modified constituent. A nucleic acid or amino acid may be detected by, e.g., hybridization to the nucleic acid (e.g., probes complementary to at least a portion of SEQ ID NOS:, 3, 4, 5 and/or 6) or to amino acids encoded into a peptide or polypeptide that comprises the amino acid sequences encoded by SEQ ID NOS: 3, 4, 5, and/or 6 with, e.g., an antibody or another agent that binds specifically to those amino acid sequences, e.g., at least a portion of a cognate protein that is known to bind to that portion of the polypeptide. The terms “staining” or “painting” are herein defined to mean hybridizing a probe of this invention to a genome or segment thereof, such that the probe reliably binds to the targeted region or sequence of chromosomal material and the bound probe is capable of being detected. The terms “staining” or “painting” are used interchangeably. The patterns on the array resulting from “staining” or “painting” are useful for cytogenetic analysis, more particularly, molecular cytogenetic analysis. The staining patterns facilitate the high-throughput identification of normal and abnormal chromosomes and the characterization of the genetic nature of particular abnormalities. Multiple methods of probe detection may be used with the present invention, e.g., the binding patterns of different components of the probe may be distinguished--for example, by color or differences in wavelength emitted from a labeled probe. One non-limiting example of staining is the binding of a BCMA specific probe that binds to the BCMA on monocytes that are obtained from a subject.

As used herein, the terms “markers,” “detectable markers” and “detectable labels” are used interchangeably to refer to compounds and/or elements that can be detected due to their specific functional properties and/or chemical characteristics, the use of which allows the agent to which they are attached to be detected, and/or further quantified if desired, such as, e.g., an enzyme, radioisotope, electron dense particles, magnetic particles or chromophore. There are many types of detectable labels, including fluorescent labels, which are easily handled, inexpensive and nontoxic.

The term “diagnosis” or “diagnostic test” for the purposes of the instant invention refers to the identification of the disease at any stage of its development, i.e., it includes the determination whether an individual has the disease or not and/or includes determination of the stage of the disease.

Antibodies against the proteins of the invention can be prepared by well-known methods using a purified protein according to the invention or a (synthetic) fragment derived therefrom as an antigen. Monoclonal antibodies can be prepared, for example, by the techniques as originally described in Kohler and Milstein, Nature 256 (1975), 495, and Galfre, Meth. Enzymol. 73 (1981), 3, which comprise the fusion of mouse myeloma cells to spleen cells derived from immunized mammals. The antibodies can be monoclonal antibodies, polyclonal antibodies or synthetic antibodies as well as fragments of antibodies, such as Fab, Fv or scFv fragments etc. As used herein, an antibody is said to “specifically bind” or “immunospecifically recognize” a cognate antigen if it reacts at a detectable level with the antigen, but does not react detectably with peptides containing an unrelated sequence, or a sequence of a different heme protein. Thus, for example, an antibody is said to be “immunospecific” or to “specifically bind” a BCMA or a BCMA variant polypeptide if it reacts at a detectable level with BCMA or a BCMA variant thereof. Affinities of binding partners or antibodies can be readily determined using conventional techniques, for example, those described by Scatchard et al. (Ann. N.Y. Acad. Sci. USA 51:660 (1949)) or by surface plasmon resonance (BIAcore, Biosensor, Piscataway, N.J.). See, e.g., Wolff et al., Cancer Res. 53:2560-2565 (1993).

Furthermore, antibodies or fragments thereof to the aforementioned polypeptides can be obtained by using methods which are described, e.g., in Harlow and Lane “Antibodies, A Laboratory Manual”, CSH Press, Cold Spring Harbor, 1988. These antibodies can be used, for example, for the immunoprecipitation and immunolocalization of the BCMA or a BCMA variant protein of the invention as well as for the monitoring of the presence of such BCMA or a BCMA variant protein for the identification of compounds interacting with the proteins according to the invention. For example, surface plasmon resonance as employed in the BlAcore system can be used to increase the efficiency of phage antibodies that bind to an epitope of the protein of the invention (Schier, Human Antibodies Hybridomas 7 (1996), 97.varies.105; Malmborg, J. Immunol. Methods 183 (1995), 7-13). Antibodies, which bind specifically to a wildtype or a variant protein can be used for diagnosing or prognosing a related disorder, e.g., cancer.

The present invention describes the discovery of novel biomarkers and novel targets for the design of new therapies for autoimmune diseases, including systemic lupus erythematosus (SLE). The present invention is based on the discovery of B cell maturation antigen (BCMA) and BCMA variant expression on SLE monocytes that can be directly associated with disease activity. The findings of the present invention can be applied to both diagnostic and therapeutic purposes. No therapeutic for blocking BCMA, BCMA variants, and BCMA-bound APRIL is currently available. The present invention enables the design of monoclonal antibodies or recombinant proteins that can block BCMA and BCMA variants as well as BCMA-bound APRIL.

In SLE, autoantibodies are both diagnostic biomarkers, which can be detected years before disease initiation, and effectors involved in disease pathogenesis. Understanding how autoreactive B cells get activated and differentiate into plasma cells is therefore a very relevant question to address.

BCMA has been known to be expressed on B cells and it plays a major role in humoral responses. BCMA contains intracellular signaling domain and thus the binding of the TNF family cytokines (BAFF and APRIL) to BCMA results in activation of B cells and further enhances B cell responses. Consistently, increased levels of serum BAFF and APRIL have been reported in SLE patients. In this invention, the present inventors first demonstrate that monocytes from SLE patients, not healthy individuals, express BCMA. The inventors also demonstrate that BCMA expressed on SLE patient monocytes can trans-present APRIL to B cells which can eventually enhance B cell responses. The percentage of APRIL+monocytes was correlated to SLE disease activity (SLEDAI). The inventors also found that exogenous APRIL can activate patient monocytes that could result in enhanced B cell responses. A further discovery indicated that SLE monocytes expressing BCMA variants have mutated forms of extracellular domains as well as variants missing transmembrane domains. Thus, BCMA and BCMA variants expressed on SLE patient monocytes could be used as biomarkers of SLE as well as targets for the design of new therapeutics for autoimmune diseases, including SLE, where BAFF and APRIL are involved in autoimmune pathogenesis.

The TNF superfamily ligands BAFF and APRIL and their receptors BAFFR, BCMA, and TACI comprise a network that is critically involved in the development and differentiation of B cells. Failure of this complex system is associated with autoimmune disease, B lymphocyte tumors, and antibody deficiency. The BAFF:BAFFR interaction is crucial for the survival of all peripheral B cell subsets as evidenced by severe B cell lymphopenia and Immoral immunodeficiency in both BAFF- and BAFFR-deficient mouse strains. In contrast, BCMA expression is highly restricted to the end stages of B cell differentiation and seems to be essential for the survival of long-lived bone marrow plasma cells (PCs). As B-cell differentiation proceeds toward plasma blasts (PBs) and PCs, the expression of TACI and of BAFF-R decreases, whereas BCMA expression increases. APRIL binds with much higher affinity to monomeric BCMA than BAFF does, suggesting a potential predominance of the APRIL-BCMA axis. In addition, proteoglycans were described as APRIL-specific binding partners and syndecan-1⁺ PCs display a highly specific binding of APRIL sensitive to inhibition by heparin. Binding of APRIL to PB proteoglycans likely contributes to promote PB survival. The functional activity of the third receptor TACI is ambiguous: while TACI−/− mice predominantly develop autoimmunity and lymphoproliferation, TACI deficiency in humans primarily manifests itself as an antibody deficiency syndrome. Expressions of those three receptors are known to be restricted to lymphocytes, mainly on B cells.

Inhibition of APRIL and BLyS, but not BLyS alone, prevented survival and/or the migration of PCs to the bone marrow. Moreover, blockade of both BLyS and APRIL, but not of BLyS alone, reduced the frequency of PCs in the spleen of SLE model mice. The presence of TACI variants in B cells has been known, but no significant association between SLE disease activity and any of TACI variants. BCMA and BAFF variants were previously reported, but association of those variants with SLE or RA was not detected. B cells from all individuals tested express those variants.

In the present invention, the inventors show that SLE monocytes, but not healthy monocytes, ectopically express different BCMA isoforms that can trans-present APRIL to B cells. APRIL, in turn, is one of the most upregulated genes induced by treatment of healthy monocytes with SLE serum in an IFN-independent manner. Addition of APRIL to SLE monocytes induces their activation and results in IL-6 and IL-10 secretion. SLE serum induced APRIL induces autocrine activation of monocytes as well as paracrine activation of B cells. Furthermore, the presence of membrane-bound APRIL on SLE monocytes correlates with disease activity according to the SLEDAI. Therefore, BCMA and BCMA variants expressed in SLE monocytes can be used as biomarkers of SLE and targets for the design of therapeutics for autoimmune diseases, including SLE.

Purification of Monocytes and B Cells: Monocytes were isolated using CD14 negative selection (Stem Cell Technologies, Canada) as per the manufacturer's instructions. B cells were isolated using CD19 negative (Stem Cell Technologies) selection as per the manufacturer's instructions. Naïve (CD19⁺IgD⁺CD27⁻) B cells were further sorted via FACSVantage (BD Bioscience) using the following Abs: CD19-APC (BioLegend), CD27-FITC (BioLegend), IgD-PE (Southern Biotech), CD3-Quantum Red (Sigma).

Monocyte/B cell co-culture: Monocytes were isolated from healthy PBMCs and PBMCs from SLE patients using CD14 negative selection (StemCell Technologies, Canada). 5×10³ monocytes were co-cultured with 4×10⁴ naïve (IgD⁺CD27⁻) sorted B cells from healthy individuals in cRPMI/10% FCS supplemented with IL-2/CpG in the presence of either 10 μg/mL control IgG (Sigma, Mo.), TACI Fc (Zymogenetics, Wash.) or BCMA Fc (Zymogenetics, Wash.). After 6 days of co-culture, differentiation was measured by an anti-CD38 Ab (Biolegened) and CFSE dilution (Invitrogen, Calif.) by flow cytometry. After 12 days of co-culture, Ig was measured in the supernatant of the co-culture, using ELISA techniques. Coating and detection Abs (Southern Biotech, Ala.). A standard curve was generated by using human reference serum (Bethyl Laboratories, Tex.).

B cell differentiation by cell membrane BCMA-bound APRIL: 293F cells were transfected with BCMA as described above. After 3 days, transfectants were fixed using Streck Cell Preservative (Streck, Md.). Transfectants were incubated at 37° C. for 30 minutes with or without 2 μg/mL APRIL trimer (Zymogenetics, Wash.), washed and then co-cultured with 4×10⁴ sorted naïve B cells (CD27⁻IgD⁻) from healthy donors using a titrating dose of transfectants in cRPMI/10% FCS supplemented with IL-2/CpG for 6 days. Differentiation was determined using an anti-CD38 Ab (Biolegened) and CFSE dilution (Invitrogen, Calif.) by flow cytometry.

BCMA Transfection monocytes and 293F cells: Monocytes were cultured for 6 hours in cRPMI prior to transfection. Monocytes were transfected using the Amaxa® Monocyte Transfection Kit (Lonza, Switzerland) as per the manufacturer's instructions, using 0.5 μg DNA and then plated at 10⁶ cells/well in a 12 well plate for 24 hours prior to analysis of surface expression. BCMA was transfected into 293 F cells in the same way for monocytes.

Activation of 293 Cells by APRIL: 293 F cells were transfected with BCMA as described above. After 3 days of culture, cells were harvested and re-plated at 10⁵ cells/well in a 96-well plate using cRPMI/10% FCS media. Cells were stimulated with 2 μg/mL of APRIL trimer for 24-72 hours. Supernatant was harvested and IL-8 production was determined using a Luminex bead-based platform (Bio-Rad, Calif.).

Serum APRIL/BAFF detection by ELISA: Blood was collected from healthy controls and SLE patients using ACD collection tubes. After centrifugation, plasma was isolated from whole blood. Serum was further isolated from plasma using Thrombin (King Pharmaceuticals, Tenn.). Serum was diluted and run on an ELISA to determine APRIL and BAFF levels in serum. Coating and detection Abs (Zymogenetics, Wash.). A standard curve was generated using recombinant human APRIL-trimer and BAFF-trimer (Zymogenetics, Wash.).

PCR Amplification of BCMA splice variants (SVs): BCMA SVs from monocytes were amplified as previously described (Smirnova et al, 2008). Briefly, monocytes were re-suspended in RLT Buffer (Qiagen) supplemented with 1% 2-ME (Sigma Aldrich). RNA was extracted using the RNAqueous® Micro Kit (Ambion) and cDNA was synthesized using the Reverse Transcription System (Promega). PCR for BCMA was performed using reagents from Promega. PCR primers (below). Cycling conditions: 40 cycles of 94° C., 45 s; 58° C., 30 s; 72° C., 60 s, [72° C. for 10 min]. Samples were run on a 4-20% TBE gel (Invitrogen). The primers used comprise: sense CAAATCCTTACGTGCCGCGAA (SEQ ID NO: 1) and anti-sense CCATTAAGCTCCCAACAGTAACCT (SEQ ID NO: 2).

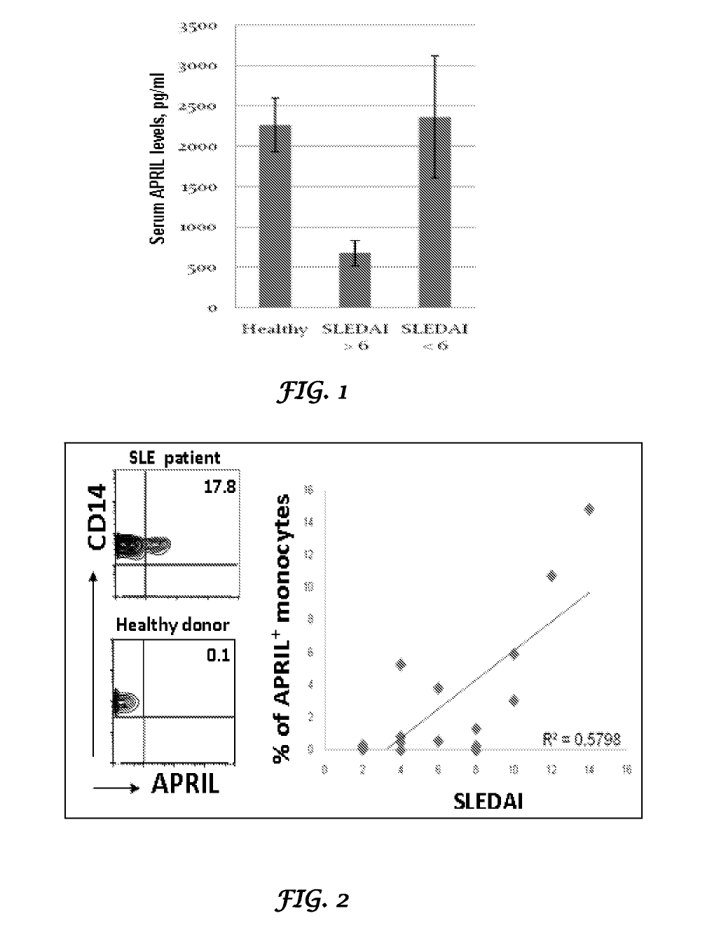

SLE monocytes express membrane-bound APRIL: The upregulation of APRIL mRNA in healthy monocytes treated with SLE serum suggests that SLE patients might display elevated serum APRIL levels. However, as FIG. 1 shows, serum APRIL levels are similar in healthy individuals and pediatric SLE patients with mild disease activity (SLEDAI<6). Intriguingly, these levels are low in SLE patients with high disease activity (SLEDAI>6). To address the dissociation between RNA and soluble protein expression, the inventors first measured monocyte surface expression of APRIL in both healthy controls and pediatric SLE patients. FIG. 2 shows that significant numbers of SLE monocytes, but not healthy monocytes, indeed express surface APRIL. To date, approximately 40% of patients tested have significant numbers of APRIL-expressing monocytes. Furthermore, the percentage of APRIL-expressing monocytes correlates with disease activity according to the SLEDAI score (FIG. 2 right panel). This suggests that APRIL-expressing SLE monocytes might contribute to enhancing auto-reactive B cell responses and increased levels of autoantibodies in these patients. The present inventors also tested proteoglycan expression on these monocytes, but they did not express significant levels of surface proteoglycans (not shown).

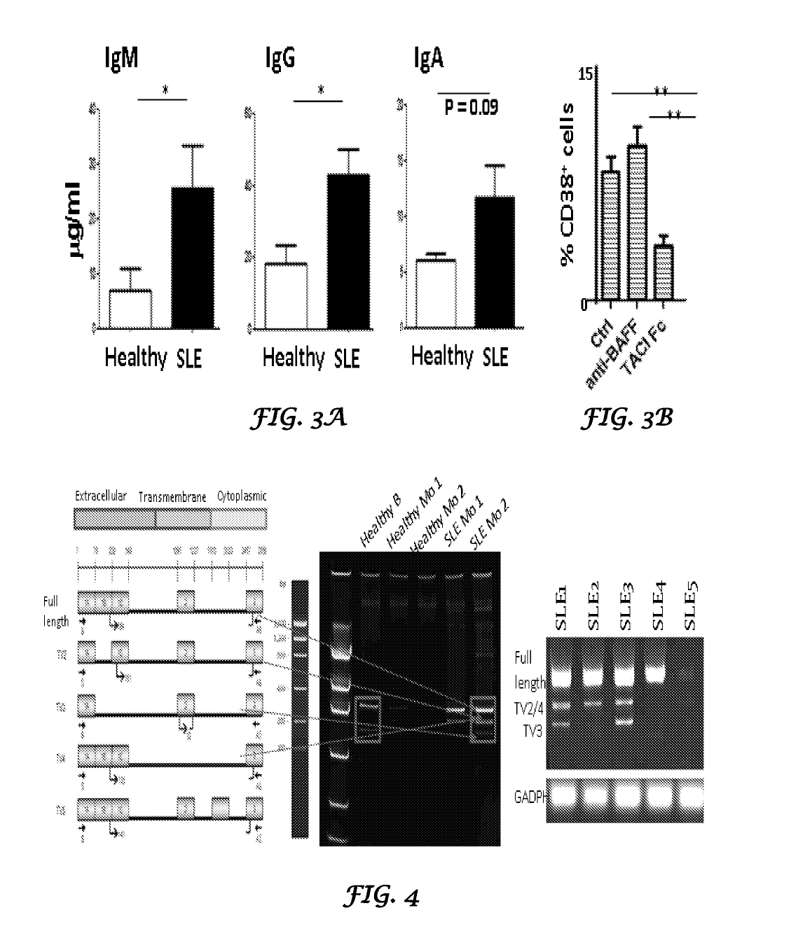

SLE monocytes stimulate B cells to secrete Igs: To test the hypothesis formulated above, freshly isolated monocytes from SLE patients and healthy donors were co-cultured with healthy donor B cells (CD19⁺IgD⁺CD27⁻) for 12 days. FIG. 3A shows that monocytes from SLE patients resulted in enhanced B cell responses, measuring total IgM, IgG, and IgA levels. These enhanced B cell responses were partially abrogated by TACI-Fc, but not anti-BAFF (FIG. 3B). Thus, it can be surmised that these enhanced B cell responses might be due, at least in part, to surface APRIL expression on SLE monocytes.

SLE monocytes express BCMA, a receptor for APRIL and BAFF: To understand the molecular mechanisms responsible for the trans-presentation of APRIL on the surface of SLE monocytes, the present inventors measured the expression levels of its receptor, BCMA, by RT-PCR. FIG. 4 shows that, as do B cells from healthy individuals do, FACS-sorted monocytes from SLE patients express full-length BCMA message. Furthermore, monocytes from some SLE patients express BCMA splicing variants 2, 3, and 4. The inventors were not able to detect variant 5. The presence of both BCMA full-length and splicing variants (variants 2, 3, and 4) was further confirmed by sequencing the corresponding bands (not shown). The inventors also cloned full-length and variant 3. Either TACI or BAFF-R was not detected in SLE monocytes. The data presented herein suggests that SLE monocytes present APRIL on their surface using BCMA, as the inventors confirmed that patient monocytes treated with heparinases could still capture and present APRIL on their surface (not shown). Expression of protein BCMA on the surface of monocytes was also confirmed by flow cytometry using commercial antibodies though the expression levels of individual variants need to be tested with variant-specific antibodies that need to be generated in the studies described herein.

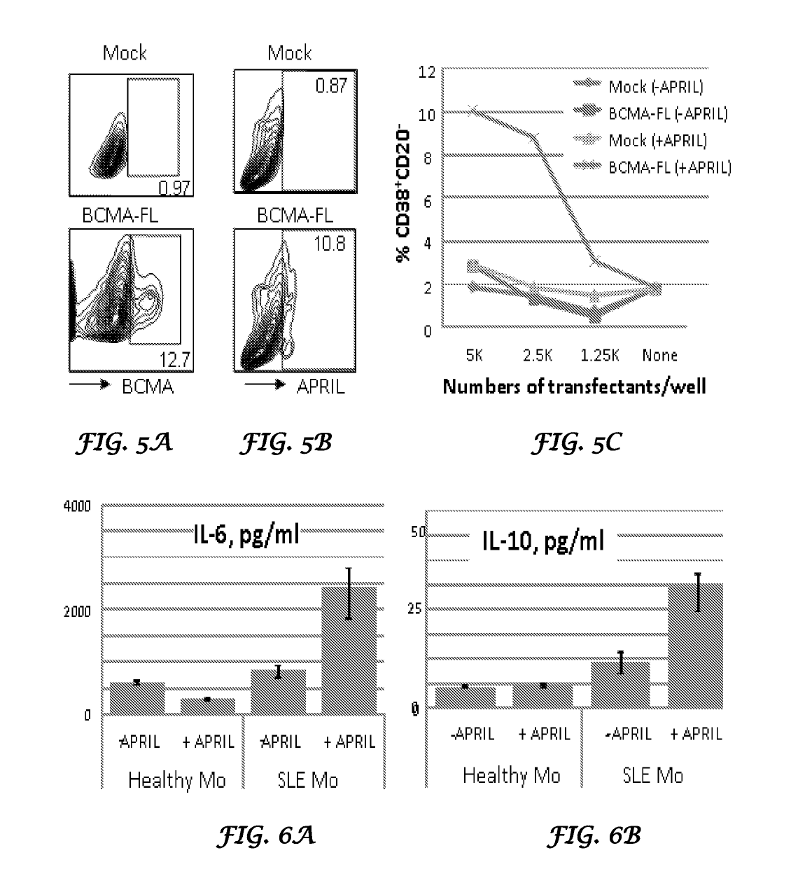

BCMA presents functionally active APRIL: The present inventors have generated 293F transfectants expressing full-length human BCMA. FIG. 5A shows that these 293F transfectants express surface BCMA. Consistently, exogenous APRIL binds to the transfectants that express full-length BCMA (FIG. 5B). To test the biological function of the membrane-bound APRIL (FIG. 5C), FACS-sorted CD27⁻IgD⁺ B cells were co-cultured with different numbers of fixed BCMA- or mock-transfectants fed with APRIL (2 μg/ml for 30 min). Transfectants were fixed and cells were vigorously washed to remove soluble APRIL before adding into the cultures. Only those BCMA-transfectants fed with APRIL induced CD27⁻IgD⁺ B cell to become CD38⁺CD20⁻PB, demonstrating that APRIL bound to BCMA is functional.

Exogenous APRIL can further activate SLE monocytes: The inventors further tested whether APRIL could activate SLE monocytes. FIGS. 6A and 6B shows that exogenous APRIL activates SLE monocytes to secrete increased amount of IL-6 and IL-10, cytokines, which can contribute to the enhanced autoreactive B cell responses. Therefore, the inventors surmised that both APRIL and BCMA expressed on patient monocytes could serve as early biomarkers of disease activity. Furthermore, it is important to test the roles of APRIL⁺ and APRIL⁻ monocytes in B cell responses in SLE. It is also valuable to test transcription profiles of the two groups of patient monocytes. This might enable discovery of new biomarkers and help design novel therapeutic approaches in the future. Anti-BLyS (Belimumab), TACI-Ig (Atacicept), and BLyS antagonist (AMG 623) are tested in SLE and anti-BLyS is currently being tested in Phase III trials. However, findings of studies conducted herein can enable the design of further advanced therapeutics for SLE.

The sequences of BCMA variants shown in FIG. 4, and are presented herein below. BCMA Full Length (SEQ ID NO: 3): aagactcaaa cttagaaact tgaattagat gtggtattca aatccttagc tgccgcgaag acacagacag cccccgtaag aacccacgaa gcaggcgaag ttcattgttc tcaacattct agctgctctt gctgcatttg ctctggaatt cttgtagaga tattacttgt ccttccaggc tgttctttct gtagctccct tgttttcttt ttgtgatcat gttgcagatg gctgggcagt gctcccaaaa tgaatatttt gacagtttgt tgcatgcttg cataccttgt caacttcgat gttcttctaa tactcctcct ctaacatgtc agcgttattg taatgcaagt gtgaccaatt cagtgaaagg aacgaatgcg attctctgga cctgtttggg actgagctta ataatttctt tggcagtttt cgtgctaatg tttttgctaa ggaagataaa ctctgaacca ttaaaggacg agtttaaaaa cacaggatca ggtctcctgg gcatggctaa cattgacctg gaaaagagca ggactggtga tgaaattatt cttccgagag gcctcgagta cacggtggaa gaatgcacct gtgaagactg catcaagagc aaaccgaagg tcgactctga ccattgcttt ccactcccag ctatggagga aggcgcaacc attcttgtca ccacgaaaac gaatgactat tgcaagagcc tgccagctgc tttgagtgct acggagatag agaaatcaat ttctgctagg taattaacca tttcgactcg agcagtgcca ctttaaaaat cttttgtcag aatagatgat gtgtcagatc tctttaggat gactgtattt ttcagttgcc gatacagctt tttgtcctct aactgtggaa actctttatg ttagatatat ttctctaggt tactgttggg agcttaatgg tagaaacttc cttggtttca tgattaaact cttttttttc ctg

BCMA-SV2 (SEQ ID NO: 4): cccccatggc taggcagtgc tccnnnnatg aatattntga cagtttgttg catgncntgc ataccttgtc aacttcgatg ttcttctaat actcctcctc taacatgtca gcgttattgt aatgcaagtg tgaccaattc agtgaaagga acgaatgcga ttctctggac ctgtttggga ctgagcttaa taatttcttt ggcagttttc gtgctaatgt ttttgctaag gaagataagc tctgaaccat taaaggacga gtttaaaaac acaggatcag gtctcctggg catggctaac attgacctgg aaaagagcag gactggtgat gaaattattc ttccgagagg cctcgagtac acggtggaag aatgcacctg tgaagactgc atcaagagca aaccgaaggt cgactctgac cattgctttc cactcccagc tatggaggaa ggcgcaacca ttcttgtcac cacgaaaacg aatgactatt gcaagagcct gccagctgct ttgagtgcta cggagataga gaaatcaatt tctgctaggt aattaaccat ttcgactcga gcagtgccac tttaaaaatc ttttgtcaga atagatgatg tgtcagatct ctttaggatg actgtatttt tcagttgccg atacagcttt ttgtcctcta annnnngnaa actctttatg ttagatatat t

BCMA-SV3 (SEQ ID NO: 5): caaatcctta cgtgccgcga agacacagac agcccccgtg tgaccaattc agtgaaagga acgaatgcga ttctctggac ctgtttggga ctgagcttaa taatttcttt ggcagttttc gtgctaatgt ttttgctaag gaagataagc tctgaaccat taaaggacga gtttaaaaac acaggatcag gtctcctggg catggctaac attgacctgg aaaagagcag gactggtgat gaaattattc ttccgagagg cctcgagtac acggtggaag aatgcacctg tgaagactgc atcaagagca aaccgaaggt cgactctgac cattgctttc cactcccagc tatggaggaa ggcgcaacca ttcttgtcac cacgaaaacg aatgactatt gcaagagcct gccagctgct ttgagtgcta cggagataga gaaatcaatt tctgctaggt aattaaccat ttcgactcga gcagtgccac tttaaaaatc ttttgtcaga atagatgatg tgtcagatct ctttaggatg actgtatttt tcagttgccg atacagcttt ttgtcctcta actgtggaaa ctctttatgt tagatatatt tctctaggtt actgttggga gcttaatgg

BCMA-SV4 (SEQ ID NO: 6): gttctcaaca ttctagctgc tcttgctgca tttgctctgg aattcttgta gagatattac ttgtccttcc aggctgttct ttctgtagct cccttgtttt ctttttgtga tcatgttgca gatggctggg cagtgctccc aaaatgaata ttttgacagt ttgttgcatg cttgcatacc ttgtcaactt cgatgttctt ctaatactcc tcctctaaca tgtcagcgtt attgtaatgc aagatcaggt ctcctgggca tggctaacat tgacctggaa aagagcagga ctggtgatga aattattctt ccgagaggcc tcgagtacac ggtggaagaa tgcacctgtg aagactgcat caagagcaaa ccgaaggtcg actctgacca ttgctttcca ctcccagcta tggaggaagg cgcaaccatt cttgtcacca cgaaaacgaa tgactattgc aagagcctgc cagctgcttt gagtgctacg gagatagaga aatcaatttc tgctaggtaa ttaaccattt cgactcgagc agtgccactt taaaaatctt ttgtcagaat agatgatgtg tcagatctct ttaggatgac tgtatttttc agttgccgat acagcttttt gtcctctaac tgtggaaact ctttatgtta gatatatt

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application No. 20090325196: Levels of BCMA Protein Expression on B Cells and use in Diagnostic Methods.

U.S. Patent Application No. 20070249530: BCMA Polypeptides and Uses Thereof

U.S. Pat. No. 6,774,106: Methods and Compositions Of Matter Concerning APRIL/G70, BCMA, BLYS/AGP-3 and TACI.

http://ukpmc.ac.uk/articlerender.cgi?artid=1004054 - ref1#ref1

What is claimed is:

1. A method for diagnosing or detecting an autoimmune condition or disorder in a human subject comprising the steps of:
identifying the human subject suspected of having the autoimmune condition or disorder;
obtaining a biological sample from the subject, wherein the biological sample comprises monocytes;
detecting the presence of a B cell maturation antigen (BCMA), BCMA variant, ligand or receptor bound BCMA, B-cell activating factor (BAFF), APRIL, TALI, BCMA-APRIL complex in or on the monocyte; and
diagnosing or detecting the autoimmune condition or disorder based on the presence of the B cell maturation antigen (BCMA), BCMA variant, ligand or receptor bound BCMA, B-cell activating factor (BAFF), APRIL, TALI, BCMA-APRIL complex.

2. The method of claim 1, wherein autoimmune condition or disorder is selected from the group consisting of auto-inflammatory diseases of the skin, allergy, sclerosis, arteriosclerosis, multiple sclerosis, asthma, psoriasis, lupus, systemic lupus erythematosis, diabetes mellitus, myasthenia gravis, chronic fatigue syndrome, fibromyalgia, Crohn's disease, Hashimoto's thyroiditis, Grave's disease, Addison's disease, Guillian Barre syndrome, scleroderma, Sjogren's syndrome, multiple sclerosis, myasthenia gravis, Reiter's syndrome, Grave's disease, alopecia areata, anklosing spondylitis, antiphospholipid syndrome, auto-immune hemolytic anemia, auto-immune hepatitis, auto-immune lymphoproliferative syndrome (ALPS), auto-immune thrombocytopenic purpura (ATP), Behcet's disease, bullous pemphigoid, cardiomyopathy, celiac sprue-type dermatitis, chronic fatigue syndrome immune deficiency syndrome (CFIDS), chronic inflammatory demyelinating polyneuropathy, cicatricial pemphigold, cold agglutinin disease, limited sclerodema (CREST syndrome), Crohn's disease, Dego's disease, dermatomyositis, discoid lupus, essential mixed cryoglobulinemia, fibromyalgia-fibromyositis, Guillain-Barre syndrome, idiopathic pulmonary fibrosis, idiopathic thrombocytopenia purpura (ITP), IgA nephropathy, juvenile rheumatoid arthritis, Meniere's disease, mixed connective tissue disease, pemphigus vulgaris, polyarteritis nodosa, polychondritis, polyglancular syndromes, polymyalgia rheumatica, polymyositis, primary agammaglobulinemia, primary biliary cirrhosis, psoriasis, Raynaud's phenomenon, rheumatic fever, sarcoidosis, scleroderma, stiff-man syndrome, Takayasu arteritis, temporal arthritis/giant cell arthritis, ulcerative colitis, uveitis, vasculitis, vitiligo, and Wegener's granulomatosis.

3. The method of claim 1, wherein the autoimmune condition or disorder is systemic lupus erythematosis (SLE).

4. The method of claim 1, wherein the BCMA, BCMA variants or both are selected from the group consisting of SEQ ID NO: 3, SEQ ID NO: 4, SEQ ID NO: 5, SEQ ID NO: 6, and any combinations thereof.

5. The method of claim 1, wherein the autoimmune disease is suspected to be systemic lupus erythematosis (SLE) and monocytes obtained from the human subject, but not normal human monocytes, express one or more BCMA isoforms selected from SEQ ID NO: 3, SEQ ID NO: 4, SEQ ID NO: 5, or SEQ ID NO: 6.

6. The method of claim 1, wherein the step of detecting is selected from detecting the presence of a nucleic acid that encodes BCMA or BCMA variants.

7. The method of claim 1, wherein the step of detecting is selected from detecting the presence of a BCMA or BCMA peptide or protein.

8. A method for diagnosing or detecting systemic lupus erythematosis (SLE) in a human subject comprising the steps of:
obtaining monocytes from a subject suspected of having SLE;
detecting a presence of a B cell maturation antigen (BCMA), BCMA variant, ligand or receptor bound BCMA, B-cell activating factor (BAFF), APRIL, TALI, BCMA-APRIL complex and modifications and combinations thereof in or on the monocytes; and
diagnosing or detecting the SLE based on the presence of the BCMA, BAFF, APRIL, TALI, BCMA-APRIL complex in or on the monocytes of the subject suspected of having the SLE.

9. The method of claim 8, wherein the normal subject is a subject not suffering from SLE, any other autoimmune condition or disorder or both.

10. The method of claim 8, wherein the BCMA, BCMA variants or both are selected from the group consisting of SEQ ID NO: 3, SEQ ID NO: 4, SEQ ID NO: 5, SEQ ID NO: 6, and any combinations thereof.

11. The method of claim 8, wherein the monocytes obtained from the human subject, but not normal human monocytes, express one or more BCMA isoforms selected from SEQ ID NO: 3, SEQ ID NO: 4, SEQ ID NO: 5, or SEQ ID NO: 6.

12. The method of claim 8, wherein the step of detecting is selected from detecting the presence of nucleic acids or amino acids.

13. A pharmaceutical composition comprising:
a therapeutically effective amount of an agent comprising a polypeptide, a protein, a peptide, an antibody or variants and modifications thereof, wherein the agent is capable of binding or interacting and thereby blocking or inhibiting one or more actions of a B cell maturation antigen (BCMA), a BCMA variant, ligand or receptor bound BCMA, a B-cell activating factor (BAFF), an APRIL, a TACI or a BCMA-APRIL complex in a monocyte; and
an optional pharmaceutically acceptable carrier.

14. The composition of claim 13, wherein the composition is adapted for use in a prophylaxis, treatment or both of one or more autoimmune disorders or conditions selected from the group consisting of auto-inflammatory diseases of the skin, allergy, sclerosis, arteriosclerosis, multiple sclerosis, asthma, psoriasis, lupus, systemic lupus erythematosis, diabetes mellitus, myasthenia gravis, chronic fatigue syndrome, fibromyalgia, Crohn's disease, Hashimoto's thyroiditis, Grave's disease, Addison's disease, Guillian Barre syndrome, scleroderma, Sjogren's syndrome, multiple sclerosis, myasthenia gravis, Reiter's syndrome, Grave's disease, alopecia areata, anklosing spondylitis, antiphospholipid syndrome, auto-immune hemolytic anemia, auto-immune hepatitis, auto-immune lymphoproliferative syndrome (ALPS), auto-immune thrombocytopenic purpura (ATP), Behcet's disease, bullous pemphigoid, cardiomyopathy, celiac sprue-type dermatitis, chronic fatigue syndrome immune deficiency syndrome (CFIDS), chronic inflammatory demyelinating polyneuropathy, cicatricial pemphigold, cold agglutinin disease, limited sclerodema (CREST syndrome), Crohn's disease, Dego's disease, dermatomyositis, discoid lupus, essential mixed cryoglobulinemia, fibromyalgia-fibromyositis, Guillain-Barre syndrome, idiopathic pulmonary fibrosis, idiopathic thrombocytopenia purpura (ITP), IgA nephropathy, juvenile rheumatoid arthritis, Meniere's disease, mixed connective tissue disease, pemphigus vulgaris, polyarteritis nodosa, polychondritis, polyglancular syndromes, polymyalgia rheumatica, polymyositis, primary agammaglobulinemia, primary biliary cirrhosis, psoriasis, Raynaud's phenomenon, rheumatic fever, sarcoidosis, scleroderma, stiff-man syndrome, Takayasu arteritis, temporal arthritis/giant cell arthritis, ulcerative colitis, uveitis, vasculitis, vitiligo, and Wegener's granulomatosis.

15. The composition of claim 13, wherein the autoimmune disorder or condition is systemic lupus erythematosis (SLE).

16. The composition of claim 13, wherein the composition is administered by an oral route, a nasal route, topically or as an injection.

17. The composition of claim 16, wherein the injection is selected from the group consisting of subcutaneous, intravenous, intraperitoneal, intramuscular, and intravenous.

18. The composition of claim 13, wherein the composition may be administered as a combination therapy with a second therapeutic agent for treating an immune-related disease, a chemotherapeutic agent, a cytotoxic agent or any combinations thereof.

19. The composition of claim 13, wherein the composition comprises a BCMA or BCMA variant antagonist.

20. The composition of claim 13, wherein the composition is an antagonist, blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof.

21. The composition of claim 20, wherein the composition is an siRNA that is an antagonist, blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof.

22. An immunosuppressive composition for suppressing an immune response, for prophylaxis, for therapy or any combination thereof against systemic lupus erythematosis (SLE) in a human subject comprising:
an agent comprising an antibody, a protein, a peptide, an antagonist or any combinations or modifications thereof, wherein the agent binds, blocks or inhibits a biological action of B cell maturation antigen (BCMA), a BCMA variant, ligand or receptor bound BCMA, a B-cell activating factor (BAFF), an APRIL, a TACI or a BCMA-APRIL complex; and
a pharmaceutically acceptable carrier, wherein the agent is comprised in an amount effective to suppress the immune response against SLE.

23. The composition of claim 22, wherein the composition is administered subcutaneously, intravenously, intraperitoneally, intramuscularly, and intravenously.

24. The composition of claim 22, wherein the composition blocks binding or inhibits the biological action of BCMA, BCMA variants or both are selected from the group consisting of SEQ ID NO: 3, SEQ ID NO: 4, SEQ ID NO: 5, SEQ ID NO: 6, and any combinations thereof.

25. The composition of claim 24, wherein the composition is an siRNA that is an antagonist, blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof.

26. The composition of claim 22, wherein the composition exerts its immunosuppressive action by blocking or inhibiting BCMA mediated activation signals to monocytes thereby inhibiting or modulating B cell responses.

27. The composition of claim 22, wherein the composition exerts its immunosuppressive action by blocking or inhibiting BCMA mediated trans-presentation of APRIL to B cells.

28. A vaccine composition comprising:
an antibody, a protein, a peptide, an antagonist or any combinations or modifications thereof capable of binding, blocking or inhibiting a biological action of B cell maturation antigen (BCMA), a BCMA variant, ligand or receptor bound BCMA, a B-cell activating factor (BAFF), an APRIL, a TACI or a BCMA-APRIL complex; and
an optional pharmaceutically acceptable carrier or an adjuvant.

29. The composition of claim 28, wherein the composition suppresses an immune response, for prophylaxis, for therapy or any combination thereof against systemic lupus erythematosis (SLE) in a human subject.

30. The composition of claim 28, wherein the composition is administered by an oral route, a nasal route, topically or as an injection.

31. The composition of claim 30, wherein the injection is selected from the group consisting of subcutaneous, intravenous, intraperitoneal, intramuscular, and intravenous.

32. The composition of claim 28, wherein the composition may be administered as a combination therapy with a second therapeutic agent for treating an immune-related disease, a chemotherapeutic agent, a cytotoxic agent or any combinations thereof.

33. The composition of claim 28, wherein the composition is an antagonist, blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof.

34. The composition of claim 28, wherein the composition blocks or inhibits BCMA mediated activation signals to monocytes thereby inhibiting or modulating B cell responses.

35. The composition of claim 28, wherein the composition blocks or inhibits BCMA mediated trans-presentation of APRIL to B cells.

36. A method for treatment, prophylaxis or both against systemic lupus erythematosis (SLE) in a human subject comprising the steps of:
identifying the human subject in need of the treatment, the prophylaxis or both against SLE; and
administering a therapeutically effective amount of an immunosuppressive composition or a vaccine comprising:
an antibody, a protein, a peptide, an antagonist or any combinations or modifications thereof capable of binding, blocking or inhibiting a biological action of B cell maturation antigen (BCMA), a BCMA variant, ligand or receptor bound BCMA, a B-cell activating factor (BAFF), an APRIL, a TACI or a BCMA-APRIL complex; and
an optional pharmaceutically acceptable carrier or an adjuvant.

37. The method of claim 36, wherein the composition or vaccine is administered by an oral route, a nasal route, topically or as an injection.

38. The method of claim 37, wherein the injection is selected from the group consisting of subcutaneous, intravenous, intraperitoneal, intramuscular, and intravenous.

39. The method of claim 36, wherein the composition or vaccine may be administered as a combination therapy with a second therapeutic agent for treating an immune-related disease, a chemotherapeutic agent, a cytotoxic agent or any combinations thereof.

40. The method of claim 36, wherein the composition comprises a BCMA or BCMA variant antagonist.

41. The method of claim 36, wherein the composition or vaccine blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof.

42. The method of claim 36, wherein the composition or vaccine may be used in a prophylaxis, a therapy or both of one or more autoimmune disorders or conditions selected from the group consisting of auto-inflammatory diseases of the skin, allergy, sclerosis, arteriosclerosis, multiple sclerosis, asthma, psoriasis, lupus, systemic lupus erythematosis, diabetes mellitus, myasthenia gravis, chronic fatigue syndrome, fibromyalgia, Crohn's disease, Hashimoto's thyroiditis, Grave's disease, Addison's disease, Guillian Barre syndrome, scleroderma, Sjogren's syndrome, multiple sclerosis, myasthenia gravis, Reiter's syndrome, Grave's disease, alopecia areata, anklosing spondylitis, antiphospholipid syndrome, auto-immune hemolytic anemia, auto-immune hepatitis, auto-immune lymphoproliferative syndrome (ALPS), auto-immune thrombocytopenic purpura (ATP), Behcet's disease, bullous pemphigoid, cardiomyopathy, celiac sprue-type dermatitis, chronic fatigue syndrome immune deficiency syndrome (CFIDS), chronic inflammatory demyelinating polyneuropathy, cicatricial pemphigold, cold agglutinin disease, limited sclerodema (CREST syndrome), Crohn's disease, Dego's disease, dermatomyositis, discoid lupus, essential mixed cryoglobulinemia, fibromyalgia-fibromyositis, Guillain-Barre syndrome, idiopathic pulmonary fibrosis, idiopathic thrombocytopenia purpura (ITP), IgA nephropathy, juvenile rheumatoid arthritis, Meniere's disease, mixed connective tissue disease, pemphigus vulgaris, polyarteritis nodosa, polychondritis, polyglancular syndromes, polymyalgia rheumatica, polymyositis, primary agammaglobulinemia, primary biliary cirrhosis, psoriasis, Raynaud's phenomenon, rheumatic fever, sarcoidosis, scleroderma, stiff-man syndrome, Takayasu arteritis, temporal arthritis/giant cell arthritis, ulcerative colitis, uveitis, vasculitis, vitiligo, and Wegener's granulomatosis.

43. The method of claim 36, wherein the composition is an antagonist, blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof.

44. The method of claim 43, wherein the composition is an siRNA that is an antagonist, blocks binding or inhibits a biological actions of BCMA, BCMA variants or both are selected from the group consisting of SEQ ID NO: 3, SEQ ID NO: 4, SEQ ID NO: 5, SEQ ID NO: 6, and any combinations thereof.

45. The method of claim 36, wherein the composition blocks or inhibits BCMA mediated activation signals to monocytes thereby inhibiting or modulating B cell responses.

46. The method of claim 36, wherein the composition blocks or inhibits BCMA mediated trans-presentation of APRIL to B cells.

47. The method of claim 36, wherein the composition comprises a BCMA or BCMA variant antagonist.

48. An isolated gene comprising SEQ ID NOS: 4, 5, or 6 or portions thereof.

49. An isolated polypeptide encoded by SEQ ID NOS: 4, 5, or 6 or portions thereof

50. A kit for detecting the presence of BCMA or a BCMA variant comprising:
one or more vials comprising a BCMA or BCMA variant detection agent adapted for detecting the presence of the BCMA or BCMA variant in or on a monocyte.

51. The kit of claim 50, wherein the BCMA or BCMA variant detection agent comprises a BCMA or BCMA binding agent.

52. The kit of claim 50, wherein the BCMA or BCMA variant detection agent comprises a BCMA or BCMA binding nucleic acid.

53. The kit of claim 50, wherein the BCMA or BCMA variant detection agent comprises a BCMA or BCMA binding antibody.

54. The kit of claim 50, wherein the BCMA or BCMA variant detection agent comprises a BCMA or BCMA cognate binding agent.

55. The kit of claim 50, wherein the BCMA or BCMA variant detection agent is defined as further comprising a chromophore, fluorophore, fluorescence resonance energy transfer (FRET) molecule, enzyme, metal particle, magnetic particle, a radiodense particle, beads, RFID, or a radioactive agent.
